(19) United States
(12) Patent Application Publication
(10) Pub. No.: US 2015/0257789 A1
(43) Pub. Date: Sep. 17, 2015

(54) SPINAL IMPLANT SYSTEM AND METHODS OF USE

(71) Applicant: Warsaw Orthopedic, Inc., Warsaw, IN (US)

(72) Inventors: Craig M. Squires, Cordova, TN (US); Mark C. Dace, Collierville, TN (US); Nikhil Kulkarni, Memphis, TN (US)

(73) Assignee: Warsaw Orthopedic, Inc., Warsaw, IN (US)

(21) Appl. No.: 14/209,388

(22) Filed: Mar. 13, 2014

(51) Int. Cl. A61B 17/70; A61F 2/44

(52) U.S. Cl. CPC A61B 17/70; A61F 2/4455; A61B 2017/00933

(57) ABSTRACT

The spinal implant includes a first end configured for connection with vertebral tissue adjacent a lamina. A second end is spaced from the first end and configured for connection with vertebral tissue adjacent a lamina. An intermediate portion includes a scaffold and a tissue barrier. Systems and methods of use are disclosed.

### TECHNICAL FIELD

The present disclosure generally relates to spinal implants for the treatment of musculoskeletal disorders, and more particularly to a spinal implant system that includes a spinal implant and a method for treating a spine.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility. For example, after a disc collapse, severe pain and discomfort can occur due to the pressure exerted on nerves and the spinal column.

Non-surgical treatments, such as medication, rehabilitation and exercise can be effective, however, may fail to relieve the symptoms associated with these disorders. Surgical treatment of these spinal disorders includes fusion, fixation, discectomy, laminectomy, laminoplasty and implantable prosthetics. For example, laminoplasty treatments may employ implants, which may include plates and bone fasteners to stabilize vertebrae and facilitate healing. This disclosure describes an improvement over these prior art technologies.

### SUMMARY

In one embodiment, a spinal implant is provided. The spinal implant includes a first end configured for connection with vertebral tissue adjacent a lamina. A second end is spaced from the first end and configured for connection with vertebral tissue adjacent a lamina. An intermediate portion includes a scaffold and a tissue barrier. In some embodiments, systems and methods are provided.

### BRIEF DESCRIPTION OF THE DRAWINGS

The present disclosure will become more readily apparent from the specific description accompanied by the following drawings, in which:

FIG. 1 is a perspective view of components of one embodiment of a system in accordance with the principles of the present disclosure;

FIG. 2 is a side view of the components shown in FIG. 1;

FIG. 3 is a plan view of the components shown in FIG. 1 disposed with vertebrae;

FIG. 4 is an axial view of the components and vertebrae shown in FIG. 1;

FIG. 5 is an axial view of components of one embodiment of a system in accordance with the principles of the present disclosure disposed with vertebrae;

FIG. 6 is a perspective view of components of one embodiment of a system in accordance with the principles of the present disclosure;

FIG. 7 is a perspective view of components of one embodiment of a system in accordance with the principles of the present disclosure;

FIG. 8 is a perspective view of components of one embodiment of a system in accordance with the principles of the present disclosure; and

FIG. 9 is a perspective view of components of one embodiment of a system in accordance with the principles of the present disclosure.

### DETAILED DESCRIPTION

The exemplary embodiments of the spinal implant system and related methods of use disclosed are discussed in terms of medical devices for the treatment of musculoskeletal disorders and more particularly, in terms of a spinal implant system that includes a spinal implant and a method for treating a spine. In one embodiment, the systems and methods of the present disclosure are employed with a laminoplasty procedure.

In one embodiment, the present disclosure provides a spinal implant system employed with a laminoplasty surgical technique that includes removing a portion of vertebral tissue, such as, for example, a portion of a spinous process and/or lamina and placing an implant adjacent and/or between the tissue adjacent a vertebra to form a bridge. In one embodiment, the spinal implant includes a plate. In one embodiment, the plate is solid. In one embodiment, the plate is perforated. In one embodiment, the plate is solid with a perforated portion. In one embodiment, the plate includes holes configured to receive bone screws to secure the plate with tissue. See also, the examples and disclosure of systems, spinal implants and methods shown and described in U.S. patent application Ser. No ______ (Attorney Docket No. C00007023.USU1) filed Mar. ______, 2014, and published as U.S. patent application Publication Ser. No. ______, on ______, the entire contents of which being incorporated herein by reference.

In one embodiment, a spinal implant is provided that maintains space between vertebral tissue where tissue is removed and is configured to receive a graft or scaffold. In one embodiment, the spinal implant includes a portion for receiving the graft or scaffold to facilitate bone growth. In one embodiment, the scaffold is integrated with the spinal implant. In one embodiment, the scaffold is independent from the spinal implant and is attached to the spinal implant in situ or prior to implantation. In one embodiment, the scaffold is configured to bridge the two sides of a lamina together.

In one embodiment, the spinal implant includes a scaffold configured for disposal with a notch in vertebral tissue, such as, for example, a lamina, transverse process, pars interarticularis, facet or spinous process, to avoid utilizing a screw to fasten the scaffold with tissue. In one embodiment, the spinal implant includes a surface adjacent the spinal cord. In one embodiment, the surface adjacent to the spinal cord is smooth to prevent irritation to the spinal cord. In one embodiment, the surface adjacent to the spinal cord is configured as a tissue barrier to prevent a fusion mass from growing into the spinal canal.

In some embodiments, the present disclosure may be employed to treat spinal disorders such as, for example, degenerative disc disease, disc herniation, osteoporosis, spondylolisthesis, stenosis, tumor and fractures. In one embodiment, the present disclosure may be employed with other osteal and bone related applications, including those associated with diagnostics and therapeutics. In one embodiment, the disclosed spinal implant system and methods may be alternatively employed in a surgical treatment with a patient in a prone or supine position, and/or employ various surgical approaches to the spine, including posterior, posterior mid-line, medial, lateral, postero-lateral approaches, and in other body regions. The spinal implant system and methods of the present disclosure may also be alternatively employed with procedures for treating the lumbar, cervical, thoracic and pelvic regions of a spinal column. The spinal implant system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended, to be limiting of the claimed disclosure. Also, in some embodiments, as used in the specification and including the appended claims, the singular forms “an,” and the include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, outer, inner, terminal (denoting position or location), left and right, posterior, anterior, and the like, are for illustrative purposes only and can be varied within the scope of the disclosure. For example, the references “superior” and “inferior” are relative and used only in the context to the other, and are not necessarily “upper” and “lower”.

Further, as used in the specification and including the appended claims, “treating” or “treatment” of a disease or condition refers to performing a procedure that may include administering one or more drugs to a patient in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (for example, preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, for example, arresting its development, or relieving the disease, for example, causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

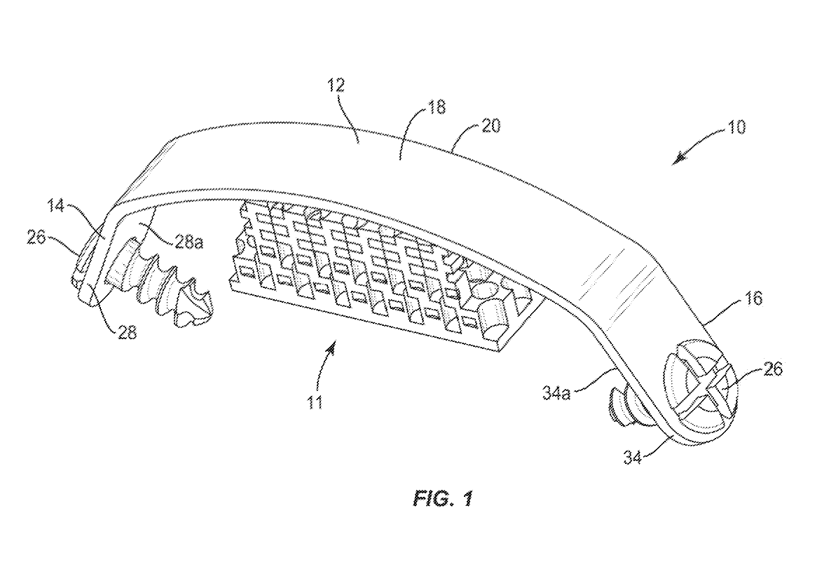

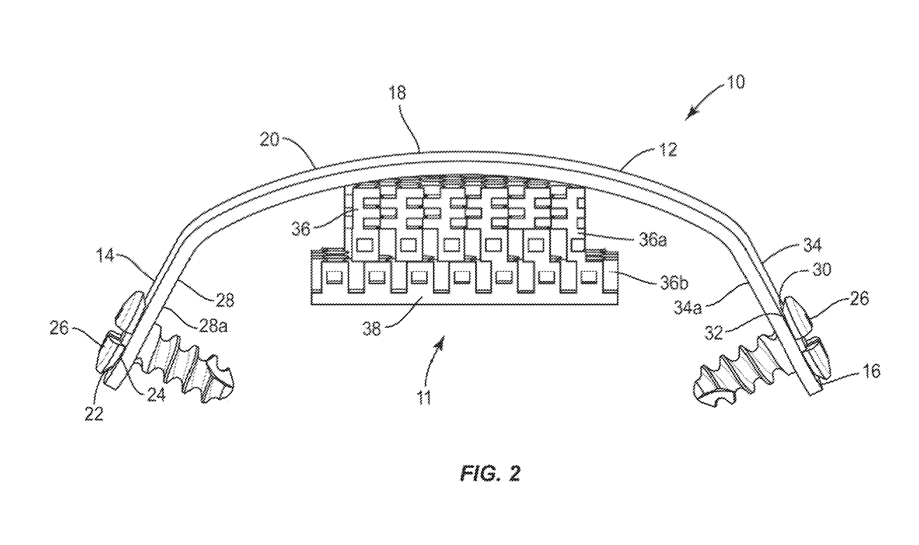

The following discussion includes a description of a spinal implant system and related methods of employing the spinal implant system in accordance with the principles of the present disclosure. Alternate embodiments are also disclosed. Reference is made in detail to the exemplary embodiments of the present disclosure, some of which are illustrated in the accompanying figures. Turning to FIGS. 1-4, there are illustrated components of a spinal implant system 10 including a spinal implant in accordance with the principles of the present disclosure.

The components of spinal implant system 10 can be fabricated from biologically acceptable materials suitable for medical applications, including metals, synthetic polymers, ceramics and bone material and/or their composites, depending on the particular application and/or preference of a medical practitioner. For example, the components of spinal implant system 10, individually or collectively, can be fabricated from materials such as stainless steel alloys, commercially pure titanium, titanium alloys, Grade 5 titanium, superelastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE® manufactured by Biologix Inc.), thermoplastics such as polyaryletherketone (PAEK) including polyetheretherketone (PEEK), polyetherketoneketone (PEEK) and polyetherketone (PEK), carbon-PEEK composites, PEEK-BaSO4 polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyimide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate such as hydroxyapatite (HA), corraline HA, biphasic calcium phosphate, tricalcium phosphate, or fluorapatite, tri-calcium phosphate (TCP), HA-TCP, calcium sulfate, or other resorbable polymers such as polyaetide, polyglycolide, polytyrosine carbonate, polycaroplaetohe and their combinations, biocompatible ceramics, mineralized collagen, bioactive glasses, porous metals, bone particles, bone fibers, morselized bone chips, bone morphogenetic proteins (BMP), such as BMP-2, BMP-4, BMP-7, rhBMP-2, or rhBMP-7, demineralized bone matrix (DBM), transforming growth factors (TGF, e.g., TGF-β), osteoblast cells, growth and differentiation factor (GDF), insulin-like growth factor 1, platelet-derived growth factor, fibroblast growth factor, or any combination thereof.

Various components of spinal implant system 10 may have material composites, including the above materials, to achieve various desired characteristics such as strength, rigidity, elasticity, compliance, biomechanical performance, durability and radiolucency or imaging preference. The components of spinal implant system 10, individually or collectively, may also be fabricated from a heterogeneous material such as a combination of two or more of the above-described materials. The components of spinal implant system 10 may be monolithically formed, integrally connected or include fastening elements and/or instruments, as described herein.

Spinal implant system 10 can be employed, for example, in laminoplasty procedures to treat patients suffering from a spinal disorder to provide stabilization and decompression. The components of spinal implant system 10 may be monolithically formed, integrally connected or include fastening elements and/or instruments, for example, as described herein.

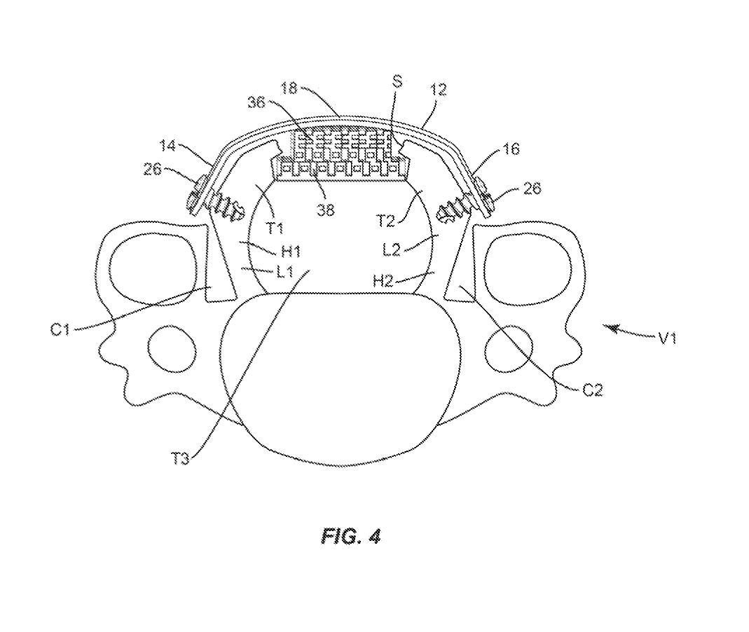

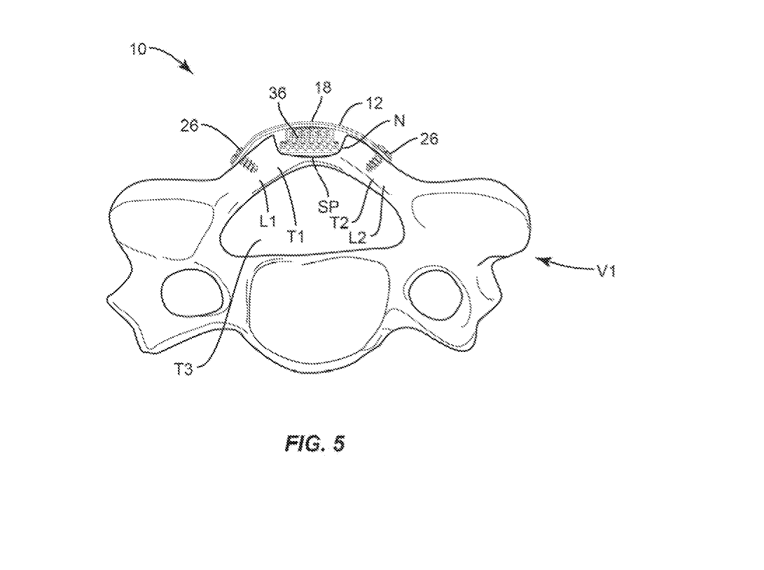

Spinal implant system 10 includes a spinal implant 11 configured for disposal with vertebral tissue in a laminoplasty procedure to treat patients suffering from a spinal disorder to provide stabilization and decompression. In some embodiments, spinal implant 11 is configured for stabilizing vertebral tissue, such as, for example, divided and/or separated lamina, transverse process, pars interarticularis, facet or spinous process portions of one or more vertebral levels, as shown and described for example with regard to FIG. 4. In some embodiments, spinal implant 11 is configured for stabilizing one or more vertebral levels via attachment with a vertebral level having removed, non-separated portions of vertebral tissue, such as, for example, a lamina, transverse process, pars interarticularis, facet or spinous process, for example, such that a cavity, relief or notch is created in the vertebral tissue, however, the tissue is not separated and spaced apart, as shown and described for example with regard to FIG. 5.

Spinal implant 11 includes a plate 12 that extends longitudinally between an end 14 and an end 16, and includes a portion 20 disposed therebetween. Plate 12 includes a wall having a substantially uniform, rectangular cross section. In some embodiments, plate 12 can have alternate configurations, such as, tubular, oval, oblong, irregular, undulating, non-uniform, variable, hollow, wire, mesh and/or tapered,

End 16 is spaced apart from end 14 and portion 20 has an arcuate configuration including a curvature that facilitates disposal of plate 12 about, in engagement and/or fixation with vertebral tissue of one or more vertebral levels. In some embodiments, portion 20 can face and/or engage adjacent, opposing, and/or distributed locations of vertebral tissue, as described herein, of a posterior, posterior mid-line, medial, lateral and/or postero-lateral portion of vertebrae. In some embodiments, portion 20 can comprise alternate configurations, such as, for example, linear or angled.

End 14 includes a flange 28 that extends from portion 20 at an angular orientation. Flange 28 includes a surface 28a oriented to face and/or engage vertebral tissue, as described herein. In some embodiments, flange 28 can face and/or engage adjacent, opposing, and/or distributed locations of vertebral tissue, as described herein, of a posterior, posterior mid-line, medial, lateral and/or postero-lateral portion of vertebrae. In some embodiments, flange 28 can comprise a member that abuts and/or engages a separated surface of an anterior facing portion of vertebral tissue, as described herein. In some embodiments, flange 28 can extend from portion 20 at various angular orientations, such as, for example, acute, obtuse and in a range of 0-360 degrees. In some embodiments, flange 28 can extend from portion 20 in a perpendicular, transverse, substantially aligned, twisted or helical orientation.

End 14 includes an inner surface 22 that defines a cavity, such as, for example, an aperture 24 configured to receive a bone fastener, such as, for example, a bone screw 26, as discussed herein. Bone screw 26 attaches flange 28 and spinal implant 11 with vertebral tissue, as described herein. In some embodiments, end 14 may include one or a plurality of cavities configured for disposal of a bone fastener. In some embodiments, end 14 may include an elongated slot for disposal of a bone fastener such that disposal of plate 11 with vertebral tissue is selectively adjustable. In some embodiments, spinal implant system 10 includes one or more of fasteners that may be engaged with vertebral tissue in various orientations, such as, for example, series, parallel, offset, staggered and/or alternate vertebral levels. In some embodiments, the fasteners may comprise pedicle screws, mono-axial screws, uni-planar screws, facet screws, fixed screws, tissue penetrating screws, conventional screws, expanding screws, wedges, anchors, buttons, clips, snaps, friction fittings, compressive fittings, expanding rivets, staples, nails, adhesives, posts, fixation plates and/or posts.

End 16 includes a flange 34 that extends from portion 20 at an angular orientation. Flange 34 includes a surface 34a oriented to face and/or engage vertebral tissue, as described herein. In some embodiments, flange 34 can face and/or engage adjacent, opposing, and/or distributed locations of vertebral tissue, as described herein, of a posterior, posterior mid-line, medial, lateral and/or postero-lateral portion of vertebrae. In some embodiments, flange 34 can comprise a member that abuts and/or engages a separated surface of an anterior facing portion of vertebral tissue, as described herein. In some embodiments, flange 34 can extend from portion 20 at various angular orientations, such as, for example, acute, obtuse and in a range of 0-360 degrees. In some embodiments, flange 34 can extend from portion 20 in a perpendicular, transverse, substantially aligned, twisted or helical orientation.

End 16 includes an inner surface 30 that defines a cavity, such as, for example, an aperture 32 configured to receive a bone fastener, such as, for example, bone screw 26, as discussed herein. Bone screw 26 attaches flange 34 and spinal implant 11 with vertebral tissue, as described herein. In some embodiments, end 16 may include one or a plurality of cavities configured for disposal of a bone fastener. In some embodiments, end 16 may include an elongated slot for disposal of a bone fastener such that disposal of plate 11 with vertebral tissue is selectively adjustable.

Spinal implant 11 includes an intermediate portion 18 configured to facilitate bone growth across at least a portion of spinal implant 11 and/or adjacent vertebral tissue connected with spinal implant, as described herein. Intermediate portion 18 includes a bone growth scaffold 36 connected with plate 12 in a configuration to grow bone and bridge vertebral tissue of one or more vertebral levels to treat patients suffering from a spinal disorder to provide stabilization and decompression.

Scaffold 36 has a stepped configuration and includes an extension 36a and a base Mb. Extension 36a and base 36b each include a plurality of cavities configured for disposal of bone growth promoting material. In some embodiments, the bone growth promoting material can include bone graft allograft, xenograft, autograft, bone paste, bone chips, Skelite®, BMP and/or a titanium mesh material, such as, for example, Trabeculite™ available from Tecomet, Wilmington, Mass. In some embodiments, the plurality of cavities may include one or more agents, as described herein. In some embodiments, extension 36a and/or base 36b may have a solid configuration, and/or scaffold 36 may include only one of extension 36a or base 36b. In one embodiment, scaffold 36 includes an outer surface having perforations that communicate with bone growth promoting material disposed with extension 36a and/or base 36b. In one embodiment, scaffold 36 has a cage configuration. In one embodiment, the plurality of cavities include pockets.

Extension 36a is directly connected to portion 20 and conforms to the curvature thereof. In one embodiment, extension 36a has a flat surface configuration oriented to face and/or engage portion 20. In one embodiment, extension 36a is spaced apart from portion 20 prior to bone growth. Base 36b has a greater width dimension relative to extension 36a and is oriented to face adjacent, opposing, and/or distributed locations of vertebral tissue, as described herein, of a posterior, posterior mid-line, medial, lateral and/or postero-lateral portion of vertebrae.

Intermediate portion 18 includes a tissue barrier 38 to prevent treatment employing spinal implant 11 and/or disposal of spinal implant 11 with vertebral tissue, as described herein, from irritating and/or undesirably engaging tissue of the spinal canal. Tissue barrier 38 is disposed adjacent scaffold 36 to prevent bone growth from the bone growth promoting material of scaffold 36 into a spinal canal. In one embodiment, tissue barrier 38 is directly connected to base 36b and includes an even surface configuration oriented to face and/or engage adjacent, opposing, and/or distributed locations of vertebral tissue, as described herein, of a posterior, posterior mid-line, medial, lateral and/or postero-lateral portion of vertebrae. Tissue barrier 38 is substantially aligned with base 36b and has a smooth surface configuration to prevent interference with tissue of the spinal canal.

In one embodiment, intermediate portion 18 is monolithically formed with plate 12. In one embodiment, intermediate portion 18 is separate and attachable with plate 12 in situ or prior to implantation. In one embodiment, tissue barrier 38 is monolithically formed with scaffold 36. In one embodiment, tissue barrier 38 is separate and attachable to scaffold 36 in situ or prior to implantation. In one embodiment, tissue barrier 38 comprises a layer of base 36b.

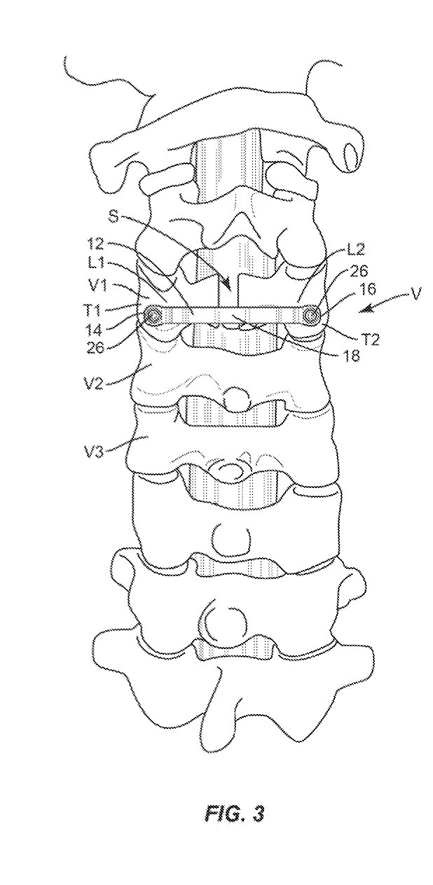

In operation, use and assembly, as shown in FIGS. 3-4, spinal implant system 10, similar to the systems described herein, is employed with a surgical procedure, such as, for example, a laminoplasty treatment of a spine of a patent including vertebrae V. Spinal implant system 10 may also be employed with other surgical procedures, such as, for example, discectomy, laminotomy, laminectomy, nerve root retraction, foramenotomy, facetectomy, decompression, and spinal, nucleus or disc replacement. For example, vertebral levels V1, V2 and V3 of vertebrae V can be removed, cut and/or weakened to open access and/or communication with a spinal canal T3 and/or spinal canal tissue, to provide space for a spinal cord. In one embodiment, spinal implant system 10 stabilizes vertebral levels V1, V2 and V3 for treatment and healing.

In use, to treat the affected section of vertebrae V, a medical practitioner obtains access to a surgical site including posterior elements of vertebrae V in any appropriate manner, such as through incision and retraction of tissues. In one embodiment, spinal implant system 10 can be used in any existing surgical method or technique including open surgery, mini-open surgery, minimally invasive surgery and percutaneous surgical implantation, whereby vertebrae V is accessed through a mini-incision, or sleeve that provides a protected passageway to the area. Once access to the surgical site is obtained, the particular surgical procedure is performed for treating the spine disorder. Spinal implant 11 is then employed to augment the surgical treatment. Spinal implant 11 can be delivered or implanted as a pre-assembled device or can be assembled in situ. Spinal implant 11 can be completely or partially revised, removed or replaced in situ. In one embodiment, one or all of the components of spinal implant system 10 can be delivered to the surgical site via manipulation and/or a free hand technique. An incision is made in the body of a patient and a cutting instrument (not shown) creates a surgical pathway for delivery of the components of spinal implant system 10 including spinal implant 11 within the patient body to adjacent vertebral level V1.

In one embodiment, as shown in FIG. 4, a cutting instrument (not shown) is employed to engage a spinous process (not shown-removed) of vertebral level V1. The spinous process is removed with the cutting instrument to form a cavity, gap or space S between lamina. L1 and lamina L2. A relief C1 is cut down a medial cortical layer of lamina L1 to create a bone hinge H1. A relief C2 is cut down the medial cortical layer of lamina L2 to create a bone hinge H2. In some embodiments, reliefs C1, C2 can include a groove, gutter or trough, and be formed using a high-speed burr drill. In one embodiment, reliefs C1, C2 have a depth of approximately 3 to 4 millimeters and a width of approximately 3 millimeters. In some embodiments, the associated ligamentum flavum, capsule, and/or veins adjacent vertebral level V1 can be separated to allow outward rotation of the separated laminae L1, L2.

Plate 12 is disposed about lamina L1 and lamina. L2 of vertebral level V1 for engagement and/or fixation with the vertebral tissue of vertebral level V1. Portion 20 faces and/or engages a posterior surface of lamina L1 and lamina L2. Lamina L1 is rotated outwardly about bone hinge H1 and lamina L2 is rotated outwardly about bone hinge H2 to enlarge the cross-sectional area of spinal canal T3. Scaffold 36, attached with plate 12 and having bone growth promoting material and/or an agent disposed therewith, as described herein, is positioned with space S between the separated surfaces of tissue T1, T2. Scaffold 36 is connected with plate 12 and disposed with lamina L1 and lamina L2 in a configuration to grow bone and bridge vertebral tissue of vertebral level V1 to provide stabilization and decompression.

In some embodiments, intermediate portion 18 can prevent the separated laminae from closing from an implant position toward an original, non-implant position. In one embodiment, intermediate portion 18 tightly abuts the spaced apart laminae.

Plate 12 is positioned in alignment for connection with vertebral level V1 for attachment of end 14 with tissue T1 of lamina L1 and end 16 with tissue T2 of lamina L2. A pilot hole or the like is formed in tissue T1 and flange 28 is disposed such that aperture 24 is aligned with the pilot hole in tissue T1. Screw 26 is disposed with aperture 24 and the pilot hole and inserted, drilled or otherwise fixed to tissue T1 to attach flange 28 with lamina L1. A pilot hole or the like is formed in tissue T2 and flange 34 is disposed such that aperture 32 is aligned with the pilot hole in tissue T2. Screw 26 is disposed with aperture 32 and the pilot hole and inserted, drilled or otherwise fixed to tissue T2 to attach flange 34 with lamina L2.

Tissue barrier 38 is connected to scaffold 36 and includes a smooth surface configuration to prevent spinal implant 11 from irritating and/or undesirably engaging tissue of spinal canal T3 adjacent vertebral level V1. Tissue barrier 38 is disposed adjacent scaffold 36 to prevent bone growth from the bone growth promoting material of scaffold 36 into spinal canal T3 adjacent vertebral level V1.

One or more of the components of spinal implant system 10 can be made of radiolucent materials such as polymers. Radio/markers may be included for identification under x-ray, fluoroscopy, CT or other imaging techniques. In some embodiments, the use of surgical navigation, microsurgical and image guided technologies may be employed to access, view and repair spinal deterioration or damage, with the aid of spinal implant system 10. In one embodiment, spinal implant system 10 may include one or a plurality of spinal implants 11 for use with a single vertebral level or a plurality of vertebral levels.

In one embodiment, spinal implant system 10 includes an agent, which may be disposed, packed or layered within, on or about the components and/or surfaces of spinal implant 11. In one embodiment, the agent may include bone growth promoting material, such as, for example, bone graft to enhance fixation of the fixation elements with vertebrae. In one embodiment, the agent may include one or a plurality of therapeutic agents and/or pharmacological agents for release, including sustained release, to treat, for example, pain, inflammation and degeneration. Upon completion of the procedure, the non-implant components, instruments and assemblies are removed and the incision(s) is closed.

In one embodiment, as shown in FIG. 5, spinal implant system 10, similar to the systems and methods described above with regard to FIGS. 1-4, is employed with a laminoplasty treatment such that spinal implant 11, as described herein, is configured for stabilizing one or more vertebral levels via attachment with vertebral level V1, which has removed, non-separated portions of vertebral tissue.

A high speed burring drill is employed to engage a spinous process SP of vertebral level V1 to form a notch N in the vertebral tissue. Spinous process SP is not separated and spaced apart. Plate 12 is disposed about lamina L1 and lamina L2, as described herein, for engagement and/or fixation with vertebral tissue T1, T2, Scaffold 36, attached with plate 12 and having bone growth promoting material and/or an agent disposed therewith, as described herein, is positioned with notch N and/or extending external to notch N adjacent vertebral level V1 in a configuration to grow bone and bridge vertebral tissue to provide stabilization and decompression. Plate 12 is fastened with vertebral level V1, as described herein. Tissue barrier 38, as described herein, is connected to scaffold 36 and/or extends external to notch N to prevent spinal implant 11 from irritating and/or undesirably engaging tissue of spinal canal T3, and/or preventing bone growth into spinal canal T3.

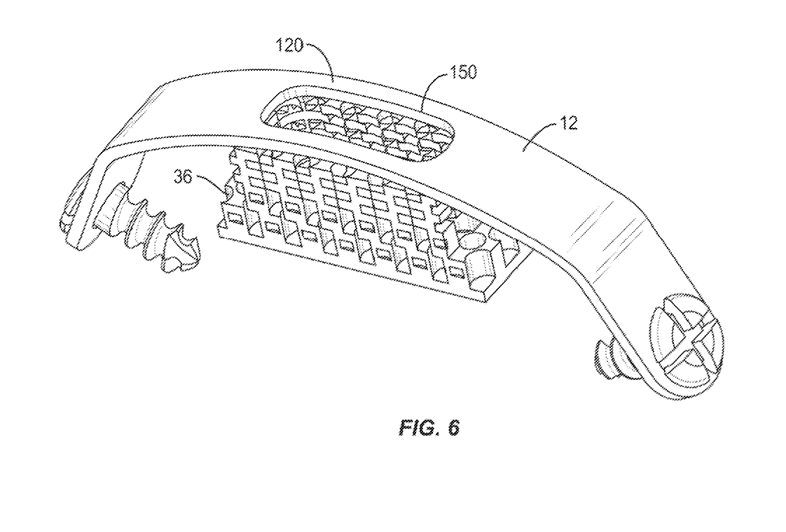

In one embodiment, as shown in FIG. 6, spinal implant system 10, similar to the systems and methods described herein, comprises spinal implant 11 having plate 12, as described herein, which includes a portion 120, similar to portion 20 described herein. Portion 120 includes at least one cavity, such as, for example, an elongated slot 150. Slot 150 is configured to facilitate communication with scaffold 36, as described herein, and facilitate bone growth.

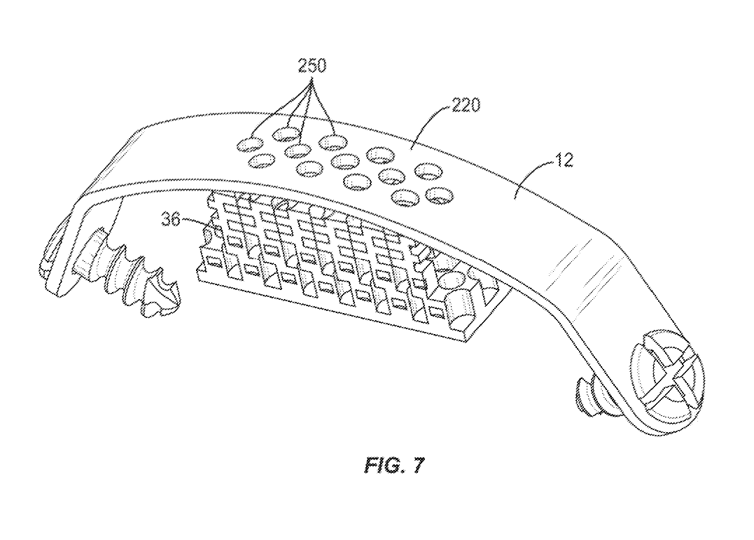

In one embodiment, as shown in FIG. 7, spinal implant system 10, similar to the systems and methods described herein, comprises spinal implant 11 having plate 12, as described herein, which includes a portion 220, similar to portion 20 described herein. Portion 220 includes a plurality of cavities, such as, for example, apertures 250. Apertures 250 are disposed in an aligned row orientation and configured to facilitate communication with scaffold 36, as described herein, and facilitate bone growth. In some embodiments, the plurality of cavities includes perforations.

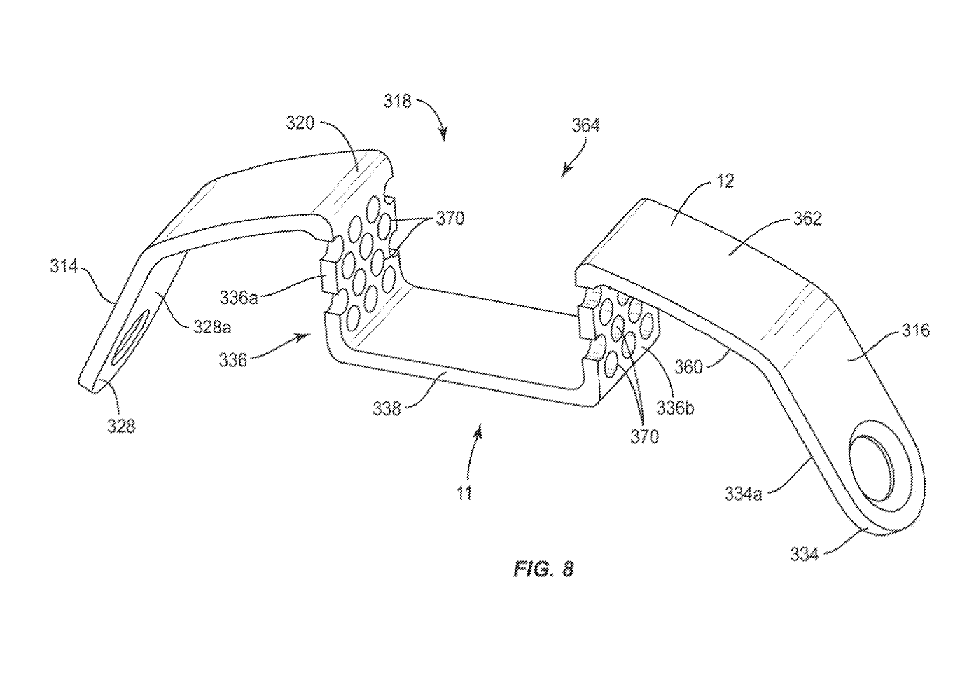

In one embodiment, as shown in FIG. 8, spinal implant system 10, similar to the systems and methods described herein, comprises spinal implant 11 having plate 12, as described herein, which includes a portion 320, similar to portion 20 described herein, having an inner surface 360 and an outer surface 362.

Spinal implant 11 includes an intermediate portion 318, similar to portion 18 described herein, configured to facilitate bone growth across at least a portion of spinal implant 11 and/or adjacent vertebral tissue connected with spinal implant, as described herein. Intermediate portion 318 includes a bone growth scaffold 336 connected with plate 12 in a configuration to grow bone and bridge vertebral tissue of one or more vertebral levels to treat patients suffering from a spinal disorder to provide stabilization and decompression.

Scaffold 336 has a substantially rectangular configuration and includes walls 336a, 336b. Walls 336a, 336b define a cavity 364 configured for disposal of bone growth promoting material, as described herein. Walls 336a, 336b each include a plurality of cavities, such as, for example, openings 370 configured for disposal of bone growth promoting material. Openings 370 facilitate communication with cavity 364 and facilitate bone growth. Cavity 364 is recessed from portion 320.

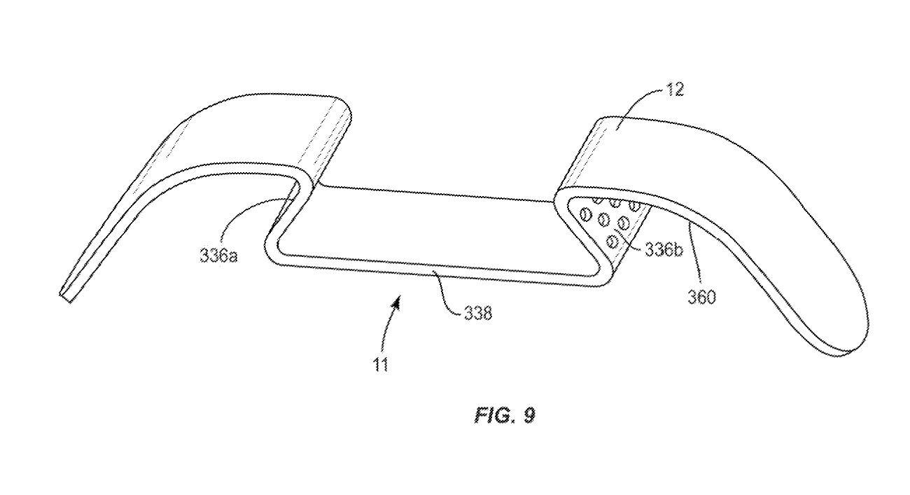

Intermediate portion 318 includes a tissue barrier 338, similar to barrier 38 described herein, to prevent treatment employing spinal implant 11 and/or disposal of spinal implant 11 with vertebral tissue, as described herein, from irritating and/or undesirably engaging tissue of a spinal canal. Tissue barrier 338 is disposed adjacent scaffold 336 to prevent bone growth from the bone growth promoting material of scaffold 336 into a spinal canal. Walls 336a, 336b are disposed in a substantially perpendicular orientation relative to inner surface 360 and tissue barrier 338. In one embodiment, as shown in FIG. 9, walls 336a, 336b are disposed in a substantially angular orientation relative to inner surface 360 and tissue barrier 338. For example, in some embodiments, wall 336a and/or wall 336b can be disposed at various angular orientations, such as, for example, acute or obtuse.

In some embodiments, plate 12 can be permanently and/or plastically deformable via an application of a compressive force on adjacent bony tissue between intermediate portion 318 including scaffold 336 and a medial surface of ends 314, 316, similar to ends 14, 16 described herein, of plate 12. Ends 314, 316 are deformable to affix spinal implant 11 with vertebral tissue, as described herein. It is envisioned that ends 314, 316 are deformable and eliminate the need for a bone screw to attach ends 314, 316 with the tissue. For example, end 314 includes a flange 328 and end 316 includes a flange 334, which extend from portion 320. Flange 328 includes a surface 328a oriented to engage vertebral tissue and flange 334 includes a surface 334a oriented to engage vertebral tissue, similar to that described herein. Flanges 328, 334 have a continuous and/or solid surface configuration. Flanges 328, 334 are manipulated, via squeezing, bending or instrument compression, for engagement with the vertebral tissue and permanently deformed to affix spinal implant 11 with vertebral tissue, as described herein. In some embodiments, surfaces 328a, 334a may include tissue fixation elements such as, for example, spikes, barbs and/or adhesives to enhance fixation.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A spinal implant comprising:
a first end configured for connection with vertebral tissue adjacent a lamina;
a second end spaced from the first end and being configured for connection with vertebral tissue adjacent a lamina; and
an intermediate portion including a scaffold and a tissue barrier.

2. A spinal implant as recited in claim 1, further comprising a plate including the ends.

3. A spinal implant as recited in claim 2, wherein the plate is monolithically formed with the intermediate portion.

4. A spinal implant recited in claim 2, wherein the intermediate portion is separate and attachable with the plate.

5. A spinal implant as recited in claim 2, wherein the intermediate portion comprises a cage recessed from an outer surface of the plate.

6. A spinal implant as recited in claim 2, wherein the plate includes a plurality of cavities that facilitate communication with the scaffold.

7. A spinal implant as recited in claim 2, wherein the plate includes an arcuate configuration.

8. A spinal implant as recited in claim 2, wherein the plate comprises a solid configuration adjacent the intermediate portion.

9. A spinal implant as recited in claim 2, wherein the plate includes at least one cavity that facilitates communication with scaffold.

10. A spinal implant as recited in claim 9, wherein the at least one cavity includes an elongated slot.

11. A spinal implant as recited in claim 1, wherein at least one of the ends comprise an angled flange.

12. A spinal implant as recited in claim 1, wherein the scaffold includes bone graft.

13. A spinal implant as recited in claim 1, wherein the scaffold includes titanium mesh material.

14. A spinal implant as recited in claim 1, wherein the tissue barrier includes an even surface configuration oriented to face vertebral tissue.

15. A spinal implant as recited in claim 1, wherein the tissue barrier includes a solid surface configuration to prevent bone growth with adjacent vertebral tissue.

16. A spinal implant comprising:
a plate including a first end configured for connection with vertebral tissue adjacent a lamina and a second end spaced from the first end, the second end being configured for connection with vertebral tissue adjacent a lamina;
a bone growth scaffold connected with an intermediate portion of the plate; and
a tissue barrier connected with the scaffold and oriented to face adjacent vertebral tissue to prevent bone growth with the adjacent vertebral tissue.

17. A spinal implant as recited in claim 16, wherein the scaffold comprises a cage recessed from an outer surface of the plate.

18. A spinal implant as recited in claim 16, wherein the plate comprises a perforated surface.

19. A spinal implant as recited in claim 16, wherein the scaffold includes titanium mesh material.

20. A spinal implant system comprising:
a first bone fastener and a second bone fastener;
a plate including a first end having an opening configured for disposal of the first bone fastener for connection with vertebral tissue adjacent a lamina and a second end spaced from the first end, the second end having an opening configured for disposal of the second bone fastener for connection with vertebral tissue adjacent a lamina;
a bone growth scaffold extending from the plate; and
a tissue barrier connected with the scaffold and oriented to face adjacent vertebral tissue to prevent bone growth with the adjacent vertebral tissue.
